(19) United States
(12) Patent Application Publication
(10) Pub. No.: US 2004/0171083 A1
(43) Pub. Date: Sep. 2, 2004

(54) Polymorphisms of the 5' region of the human 5-HT1A gene, associated proteins of the 5' region and a diagnostic test for major depression and related mental illnesses

(76) Inventors: Paul Albert, Ottawa (CA); Sylvie Lemonde, Cantley (CA)

Correspondence Address: SHERIDAN ROSS PC, 1560 BROADWAY, SUITE 1200, DENVER, CO, 80202

(21) Appl. No.: 10/635,855

(22) Filed: Aug. 5, 2003

Related U.S. Application Data
(63) Continuation-in-part of application No. 09/430,412, filed on Oct. 29, 1999.
(60) Provisional application No. 60/106,375, filed on Oct. 30, 1998.

(51) Int. Cl. C12Q 1/68; G01N 33/53; G01N 33/567

(52) U.S. Cl. 435/7.2; 435/6

(57) ABSTRACT

Clinical response to antidepressant compounds correlates with a selective down-regulation of presynaptic 5-HT1A receptors in serotonergic raphe neurons. Thus regulation of the 5-HT1A receptor gene could play a crucial role in the treatment or etiology of major depression. The promoter and repressor activities of the human 5-HT1A receptor gene have been examined. The analysis of the 5′-flanking regions of the 5-HT1A receptor gene has revealed a segment located between about −3438 and about −393 bp upstream from the initiator ATG that mediates cell-specific repression of the gene that is greater in cells that do not express the 5-HT1A receptor. The sequence of part of this region in patients with major depression was examined and a polymorphic C-G change located at −1019 bp (numbered earlier as −1017) was identified, which is associated with major depression. Thus, this sequence can be used as a genetic marker for major depression and related mental illnesses. Proteins that bind to the DNA at the −1019 locus have been identified. Such proteins that bind to this DNA region, for example the transcription factors NUDR/DEAF-1/suppressin and HES-5, are important targets for the development of therapeutic compounds for the treatment of major depression and related mental illness that involve the serotonin system. In addition the promoter region from about −393 to the initiator ATG displays glucocorticoid-mediated repression.

### FIELD OF INVENTION

The present invention relates to a DNA sequence of the 5′ flanking region of the 5-HT1A receptor gene, from about −3438 to about −393, wherein said sequence contains a polymorphism that results in a reduction of protein-DNA interactions. This invention further relates to proteins, which bind to this region and the use of said proteins, as targets, to develop therapeutics to treat depression and related illnesses that involve the serotonin system. This invention also relates to a diagnostic or prognostic test for mental illnesses, and other conditions that involve the serotonin system, using the novel DNA sequence as a genetic marker. This invention also relates to a glucocorticoid-responsive element located from about −393 to the ATG initiation codon of the 5-HT1A receptor gene.

### BACKGROUND OF THE INVENTION

Serotonin (5-HT), a key neurotransmitter in the central nervous system, is believed to play a role in various cognitive functions such as sleep, pain perception, depression, learning and anxiety (Blier et al., 1990; Jacobs and Azmitia, 1992; Mongeau et al., 1997). Neurons of the raphe nuclei which release serotonin have project axons widely throughout the brain to innervate a variety of nuclei (Tork, 1990). The activity of the raphe nucleus is controlled in part by inhibitory somatodendritic 5-HT1A autoreceptors. The serotonin 1A (5-HT1A) receptor belongs to the seven-transmembrane G-protein coupled receptor superfamily (Hoyer et al., 1994). Its activation inhibits adenylyl cyclase activity, increases K+ conductance causing a decrease in action potential frequency, and decreases the opening of voltage-dependent calcium channels (Penington and Kelly, 1990; Penington et al., 1993; Zgombick et al., 1989). An important function of 5-HT1A autoreceptors in the raphe nuclei is thus to control the frequency of action potential firing. Increase in action potential frequency leads to serotonin release at the cell body, which activates the 5-HT1A receptor to decrease raphe firing and reduce the release of serotonin, as part of a negative feed-back loop (Albert et al., 1996).

Recent studies have suggested that the level of expression of 5-HT1A autoreceptors may play a role in the treatment and possibly the etiology of major depression (Albert et al., 1996; Blier and de Montigny, 1994; Mongeau et al., 1997). Antidepressant compounds (monoamine oxidase inhibitors, tricyclic reuptake inhibitors, and especially serotonin-selective reuptake inhibitors (SSRIs)) act to enhance serotonin release by inhibiting its elimination. These compounds are effective in the treatment of a variety of mental illnesses including major depression, bipolar depression, generalized anxiety disorder, and obsessive compulsive disorder, but 2-3 weeks of treatment are required before clinical improvement may be observed (Charney et al., 1990). Acute treatment with antidepressants to enhance synaptic serotonin levels leads to inhibition of the firing rate of raphe neurons via activation of 5-HT1A autoreceptors, which prevent enhancement of serotonin release (FIG. 2). Chronic (2 weeks) treatment with serotonin uptake inhibitors (eg. fluoxetine) and selective 5-HT1A partial agonists (eg. buspirone) results in a selective downregulation of presynaptic (eg. raphe) but not postsynaptic 5-HT1A receptors (hypothalamus, cortex, hippocampus) (Fanelli and McMonagle-Strucko, 1992; Welner et al., 1989). Desensitization of the 5-HT1A autoreceptor results in restoration of raphe firing rate and enhanced serotonergic neurotransmission (FIG. 2) that correlates with behaviourial improvement induced by antidepressant treatments.

As longterm regulation of the 5-HT1A receptor is implicated in major depression, we have investigated the promoter of the human 5-HT1A receptor gene to characterize and identify specific loci associated with depression. Changes in gene expression persist for days to weeks, and could underlie the down-regulation of 5-HT1A receptors by antidepressant compounds over the 2-week treatment period.

### SUMMARY OF THE INVENTION

The present invention relates to a proximal ubiquitous promoter region flanked by a repressive region containing several elements of interest including the RE-1 element (Schoenherr and Anderson, 1995) and a poly GT dinucleotide repeat also present in the equivalent region of the rat gene. Using PCR and DNA sequence analysis of the 5-HT1A receptor gene from blood samples of depressed patients, we have further identified in the repressor region a polymorphic C-G conversion that is located at −1019 bp (numbered earlier as −1017) upstream of the initiation ATG codon. Depressed patients were about twice as likely as the controls to have the homozygous G(−1019) genotype, whereas suicide victims were four times as likely to carry the same genotype. Within the proximal 5′-flanking region of the human 5-HT1A receptor gene a novel glucocorticoid responsive region that suppresses reporter gene expression has been identified in the present invention.

The present invention thus relates to a DNA sequence of the 5′ flanking region of the 5-HT1A receptor gene, from about −3438 to about −393 (SEQ ID NO:1), wherein said sequence contains a polymorphism which results in an inhibition of protein-DNA interactions. The wild-type sequence of the human 5-HT1A receptor gene is deposited in Genbank #AC 008965. More specifically this invention relates to a DNA sequence comprising a polymorphic C-G change at position −1019 of the 5-HT1A receptor gene and to DNAfragments containing the −1019 locus.

This invention also relates to a diagnostic or prognostic test for mental illnesses that involve the serotonin system using the novel DNA sequence of the present invention, including the −1019 locus as a genetic marker. Kits for conducting the tests of the present invention are also included within the scope of this invention.

In this aspect of the invention there is provided a method for detecting depression and related mental illnesses comprising the steps of:

selecting primers to amplify a DNA region from the repressor of the 5-HT1A receptor gene;

amplifying the DNA region from the repressor of the 5-HT1A receptor gene; and

determining the sequence of the DNA region from the repressor of the 5-HT1A receptor gene, whereby identifying a mutation in said DNA region, wherein said mutation results in a reduction in repressor function leading to enhanced 5-HT1A receptor expression, which is correlated to depression and related mental illnesses.

This invention further relates to proteins, which bind to this region and the use of said proteins to develop therapeutics to treat depression and related illnesses that involve the serotonin system. More specifically this invention relates to proteins which bind to a DNA fragment including the −1019 locus.

In this aspect of the invention there is provided a method of identifying a protein which binds to a DNA region from the repressor of the 5-HT1A receptor gene comprising the steps of:

using an oligonucleotide from the repressor region of the 5-HT1A receptor gene to screen a cDNA expression library;

identifying a protein that binds to the oligonucloetide; and

cloning cDNA's of the proteins that bind the oligonucleotide.

In a further aspect of this invention there is provided a method for identifying a therapeutic effective agent to treat depression and related mental illnesses, wherein said agent binds to a DNA sequence containing a mutation in the repressor region of the 5-HT1A receptor gene and said mutation results in a reduction in repressor function leading to enhanced 5-HT1A receptor expression, comprising the steps of:

screening a library of test agents;

identifying an agent that binds to the DNA sequence.

This invention also relates to antibodies to the proteins that bind to the DNA region containing the polymorphism. Specially, the invention relates to antibodies that bind to the transcription factors NUDR/DEAR-1/suppression or HES-5.

This invention also includes a method of identifying novel therapeutics using a DNA sequence containing a mutation in the repressor region of the 5-HT1A receptor gene, wherein said therapeutics will modify the protein-DNA binding at the site, which is predicted to be reduced in patients suffering from depression and related illnesses.

This invention also includes a glucocorticoid-responsive element located between −393 and the initial ATG codon.

### BRIEF DESCRIPTION OF THE DRAWINGS

These and other features of the invention will become more apparent from the following description in which reference is made to the appended drawings wherein:

FIG. 1a and 1b shows the promoter region of human 5-HT1A from −3438 to −393. The position of the polymorphism at −1019 is shown by an arrow.

FIG. 2 shows acute and chronic actions of antidepressants on the serotonin system. Acutely, antidepressants that block the presynaptic 5-HT reuptake transporter (eg. SSRIs) inhibit serotonergic firing via recurrent or dendro-dendritic activation of 5-HT1A autoreceptors. After 3 weeks of treatment, a reduction in the number of 5-HT1A autoreceptors via homologous desensitization is observed: this disinhibits the serotonergic neurons, enhancing action potential firing rate, and increasing serotonergic neurotransmission. See text for discussion.

FIG. 3 shows regions of cell-specific transcriptional repressor activity of the 5-HT1A receptor gene. The transcription start site of the human 5-HT1A receptor gene is shown by the solid arrow and restriction sites used to produce the luciferase reporter constructs are indicated as well. Numbers indicate distance from the initial coding ATG codon. Luciferase activity of each reporter construct is normalized that of basal activity of the vector (pGL3-Basic), with pGL3-Promoter plasmid as a positive control. Activities were obtained from eleven (SN-48), four (HEK-293) or thirteen (RN46A) separate experiments in which triplicate transfections were performed and corrected for transfection efficiency with a co-transfected pCMV-βGal plasmid. Data are presented as mean±SD.

FIG. 4 shows glucocorticoid-induced suppression of 5-HT1A gene in SN-48 (FIG. 4A) cells, but not RN46A (FIG. 4B) cells. Numbers indicate distance from the initial ATG codon (see FIG. 3). Luciferase activity of each reporter construct is normalize to that of the vector (pGL3-Basic). Activities were obtained from thirteen separate experiments in which triplicate transfections were performed and corrected for transfection efficiency with a co-transfected pCMV-βGal plasmid. Error bars indicate mean±SD, n=13.

FIG. 5 shows the detection of the G-C polymorphism at −1019 bp of the 5-HT1A gene in human blood samples. A 716-bp fragment in the repressor region of the human 5-HT1A promoter was amplified by PCR and sequenced. Shown is partial DNA sequence from PCR products of three different depressed patients that revealed: 5A, the homozygous C(−1019) sequence; 5B, the heterozygous sequence with both C and G at the (−1019) site; and 5C, the homozygous G(−1019) sequence.

FIG. 6 shows the association of nuclear proteins with the polymorphic site of the 5-HT1A receptor gene. EMSA was done using a 26-bp probe that includes the C(−1019)G polymorphism of the human 5-HT1A receptor gene associated with depression. Labeled 26-bp oligonucleotide probes were present in samples as the C(−1019) or the G(−1019) allele as indicated. Unlabelled 26-bp oligonucleotides (26bp-C or 26bp-G) or unrelated E2F oligonucleotides (E2F) were added at 100-fold or 1-,2-,3- or 4-fold molar concentration to the incubation as indicated. (6A) Using RN46A nuclear extracts, a single specific complex was observed (arrowhead) that showed preferential binding to the 26bp-C oligonucleotide. (6B) Decreased competition was observed with the 26 bp-G oligonucleotide, which required a two-fold molar excess to start competing with the 26bp-C probe for RN46A nuclear extracts. (6C) Nuclear extracts from rat brain tissues, including raphe/midbrain, cortex (Ctx) and hippocampus (Hip) showed specific binding to the labeled 26 bp-C oligonucleotide (arrowhead). (6D) Yeast one-hybrid cloning of binding proteins specific for the C(−1019) palindrome allele. Six copies of the C(−1019) or G(−1019) alleles of the 26-bp element of the 5-HT1A receptor gene were integrated in the yeast genome 5′ to the LacZ gene. The two yeast strains generated were non-transformed (Control) or transformed with plasmid DNA from the indicated cDNA clones (76D, 18C, or 33B). Trans-activation was measured by β-galactosidase activity in a plate assay (left), and by quantitative spectrophotometry to calculate the activity ratio of C/G (right), expressed as mean±SD (n=2).

FIG. 7 shows the trans-repression at the palindrome of the 5-HT1A receptor gene by NUDR and HES-5: differential sensitivity to the C(−1019)G polymorphism. The C(−1019) or G(−1019) alleles of each 5-HT1A reporter construct were transfected into 5-HT1A receptor-negative HEK 293 or receptor-positive raphe RN46A cells, as indicated. Cells were cotransfected with vector (pcDNA3), NUDR or HES-5 expression plasmids, as indicated. Luciferase activity was assessed as described (Ou et al., 2000) (adjusted luciferase activity) or normalized to control transfections as indicated. Data are presented as mean±SD of triplicate samples from experiments that were repeated at least twice, except (7C), which is the mean±SD of five independent experiments. Significance compared to control or as indicated: *P<0.05, **P<0.005,***P<0.0005 (7A, 7B) Repression of the human 5-HT1A promoter (−1128-bp to the initiation ATG) by NUDR and HES-5 in HEK 293 (7A) and RN46A cells (7B). Cotransfection of NUDR or HES-5 repressed the C(−1019) allele, 5-HT1A(C), but lacked significant activity at the G(−1019) allele, 5-HT1A(G). (7C) C-G sensitive repressor activity of the polymorphic region at the SV40 promoter. A 209-bp fragment spanning the C(−1019)G polymorphism was placed upstream of the SV40 promoter in pGL3P (209 bp-C or 209 bp-G) in the forward or reverse orientation as indicated. (7D) Repression of SV40 promoter at a hexamer of 26-bp elements in RN46A cells. Six copies of the 26-bp element (26 bp-C(6) or 26 bp-G(6)) were placed upstream the SV40 promoter in the pGL3P plasmid. The C-G change blocked basal repression and repression by NUDR but not HES-5. (7E, 7F) Repression of TK promoter at a single 26-bp element by NUDR and HES-5. Both alleles of the 26-bp element were cloned upstream (5′) of the TK promoter (26 bp-C(TK) or 26 bp-G(TK)) and cotransfected with vector (pcDNA3), NUDR, or HES-5 plasmids in HEK 293 (7E) or RN46A cells (7F).

FIG. 8 shows the presence of NUDR bound to the C(−1019) palindrome in RN46A nuclear extracts. Electrophoretic mobility shift assay (EMSA) was done with the 26 bp-C probe using recombinant NUDR or RN46A nuclear extracts as indicated. Unlabelled 26-bp oligonucleotides (26 bp-C) or unrelated E2F oligonucleotides (E2F) were added at 100-fold molar excess to the incubation as indicated. (8A) In vitro-transcribed and—translated recombinant NUDR bound specifically to the 26 bp-C probe. (8B) Binding of endogenous NUDR to the C(−1019) palindrome in RN46A nuclear extracts. A major specific complex was observed (left arrowhead) that was supershifted (right arrowhead) upon incubation with anti-NUDR antibody. (8C) Neither anti-HES-5 antibody nor rabbit pre-immune serum produced a supershift of the protein-DNA complex in RN46A cells. (8D) Localization of NUDR binding site within the proximal 5-HT1A promoter region by DNase I protection assay. A 405-bp DNA probe spanning the region between −723 and −1128 was treated with the indicated units of DNase I in the absence (a) or presence of 10 μg (b) or 20 μg (c) of raphe/midbrain nuclear extracts, or in the presence of recombinant NUDR protein (R). Midbrain extracts and NUDR protected a region centered between −1016 and −1019, corresponding to the TTCG NUDR recognition sequence and the polymorphic site (C(−1019)G). The nucleotide position within the 5-HT1A 5′-flanking sequence is marked adjacent to the sequencing reaction (G).

FIG. 9 shows that the NUDR protein is expressed in 5-HT1A receptor-positive cells and brain regions and regulates 5-HT1A protein expression. Western blot analysis of nuclear extracts from RN46A cells (1), adult rat raphe/midbrain (2), cortex (3) and hippocampus (4) using anti-NUDR (9A) or anti-HES-5 (9B) antibodies. A common 33-kDa band on Coomassie-stained gel is shown as a loading control. NUDR was expressed in nuclear extracts from RN46A cells and rat brain tissues while HES-5 expression was restricted to RN46A cells. (9C) Western blot analysis and [³H]-8-OH-DPAT binding for RN46A cells stably expressing NUDR (clones 11, B, and 15). Left panels: NUDR reduced 5-HT1A binding and 5-HT1A protein expression. β-actin immunoreactivity was tested to confirm equal loading. Right panel: Western blot analysis showing 5-HT1A receptor immunoreactivity in myoblast L6 cells transfected with the rat 5-HT1A expression vector but not with pcDNA3 vector, as indicated. (9D) Strong NUDR immunostaining was detected in the dorsal raphe nucleus (DRN) and the CA2, CA3 and dentate gyrus (DG) of the hippocampus. (9E) Colocalization of NUDR and 5-HT1A receptor or 5-HT in RN46A cells (1), primary cultures of embryonic hippocampal (2) and cortical (3) cells and dorsal raphe nucleus (4). In the dorsal raphe nuclei, some cells (indicated by an arrow) that stained for NUDR displayed weak 5-HT1A receptor or 5-HT staining.

FIG. 10 shows the co-staining of NUDR with neuronal markers and specificity of immunohistochemical staining. (10A) Primary cultures of cortical (1) and hippocampal (2) cells were co-stained with antibodies to NUDR and neuronal markers MAP2 and TuJ 1, demonstrating the presence of NUDR in neurons. (10B) Specificity of 5-HT,5-HT1A and NUDR staining in raphe nuclei. Immunofluorescence was visualized through the green and red channels as indicated using primary 5-HT1A and NUDR antibodies in the absence of secondary antibodies, no primary antibodies in the presence of both secondary antibodies as indicated. Specific immunoreactivity through the green but not the red channel using anti-5-HT or anti-5-HT1A antibodies in the presence of both secondary antibodies. NUDR immunoreactivity observed through the red channel was absent in the green channel using both secondary antibodies. NUDR immunofluorescence in the presence of hNUDR (36-51) blocking peptide (100 μg) is greatly reduced.

FIG. 11 shows the decreased response to antidepressant treatment in G(−1019)G depressed patients. The efficacy of various antidepressant drugs in the treatment of major depressive disorder was examined in 119 patients who completed the drug trial study. Depressed individuals were administered one of the following daily oral doses: fluoxetine (selective serotonin reuptake inhibitor, 75-150 mg); nefazodone/pindolol (weak serotonin-noradrenalin reputake blocker, potent 5-HT2 receptor antagonist/5-HT1A antagonist, (3×2.5 mg /20-40 mg); fluoxetine/pindolol (10-20 mg/4-16 mg); NKP608A (Novartis NK1 receptor antagonist, 0.55 or 25 mg); BIMT 17 (5-HT1A agonist and 5-HT2A antagonist, 2×20-50 mg or 20-100 mg), for a period of at least 3 weeks. Although no difference in the severity of depression was noted across genotypes before treatment according to the HAMD17 scores, when patients on any antidepressant treatment were pooled, there was a statistically significant (P=0.0479) reduction in effectiveness amongst individual carrying the G(−1019)G genotype compared to depressed patients carrying the C(−1019)C genotype. The numbers of patients in each genotype groups are as indicated.

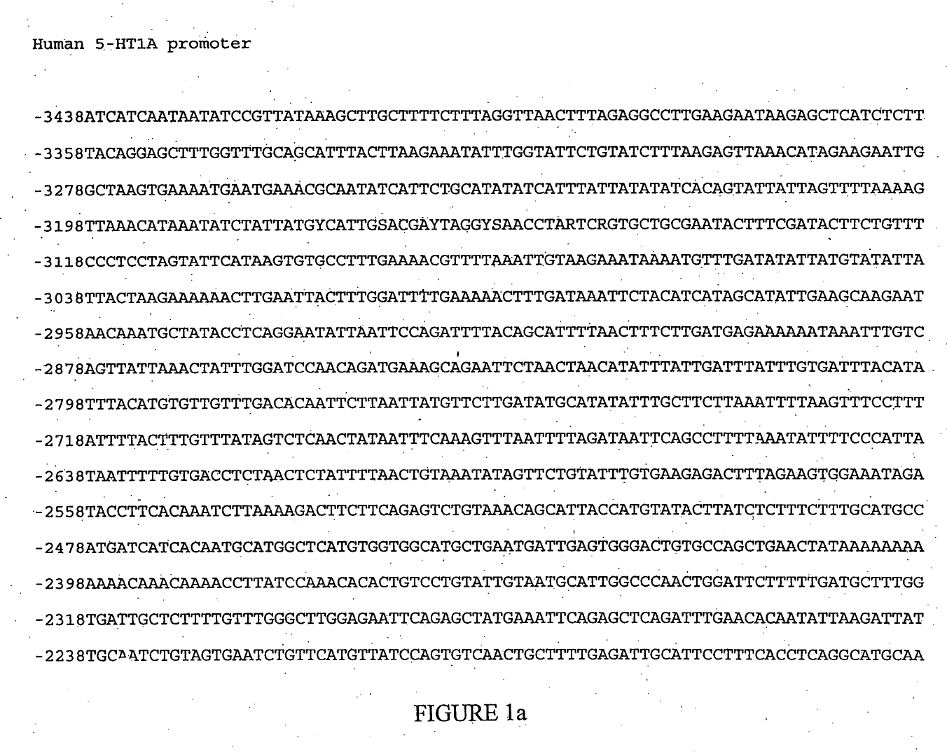

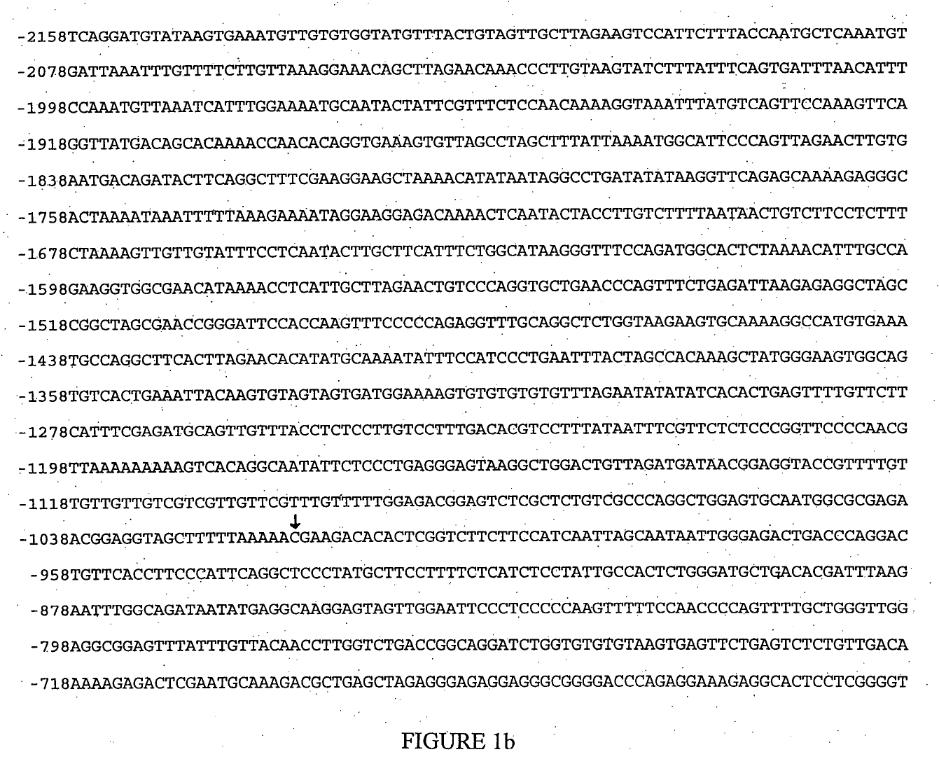

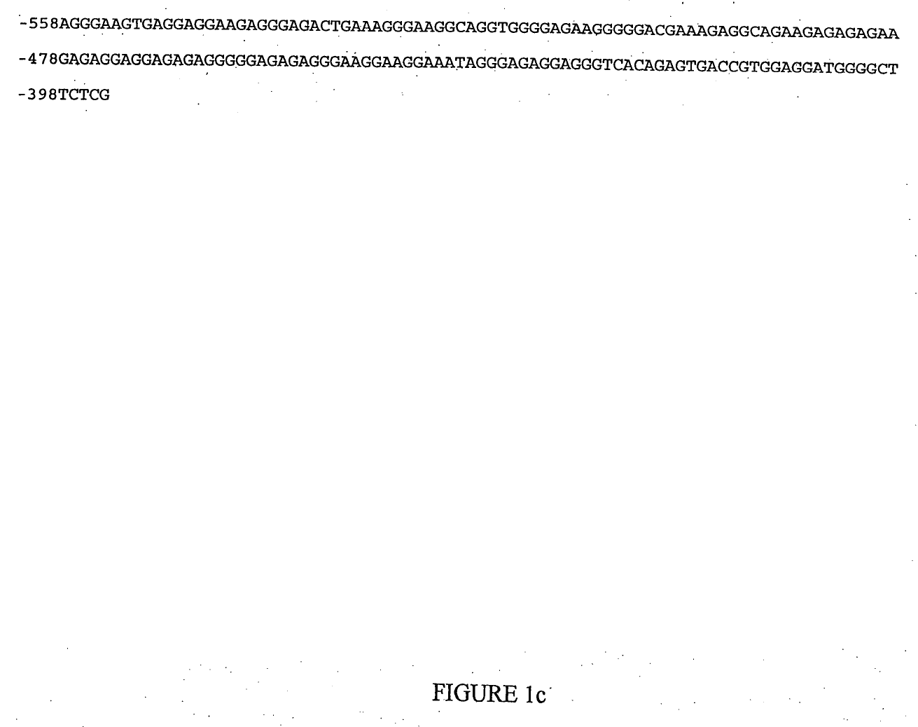

### DESCRIPTION OF PREFERRED EMBODIMENT

The present invention relates to a DNA sequence of the 5′ flanking region of the 5-HT1A receptor gene, from about −3438 to about −393, wherein said sequence contains a polymorphism that results in an inhibition of protein-DNA interactions. The novel DNA sequence can be used as a genetic marker in a diagnostic or prognostic test for mental illnesses that involve the serotonin system. This invention further relates to proteins, which bind to this region and the use of said proteins, as targets, to develop therapeutics to treat depression and related illnesses that involve the serotonin system. This invention also relates to a glucocorticoid-responsive element located from about −393 to the ATG initiation codon of the 5-HT1A receptor gene.

In the context of the present invention a mutation includes any modification of the DNA sequence. Such modifications include but are not limited to single or multiple base pair changes, inversion, deletions or insertion.

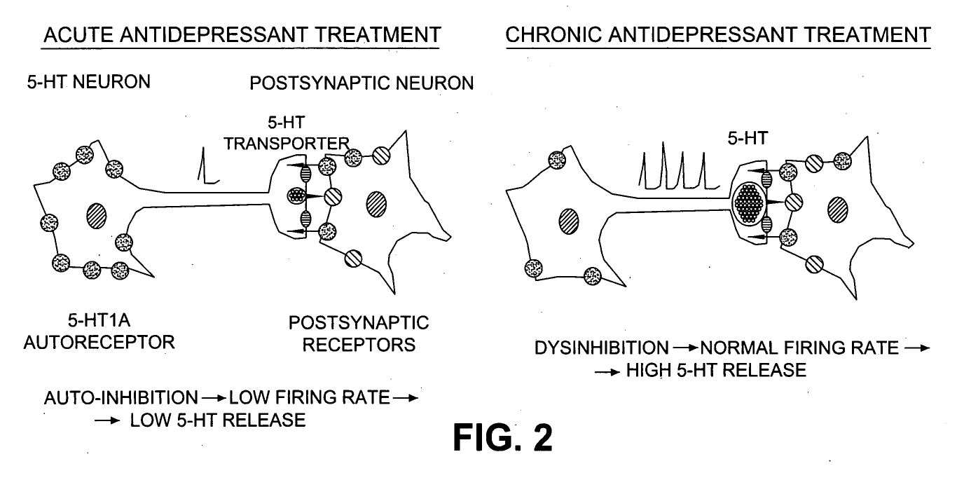

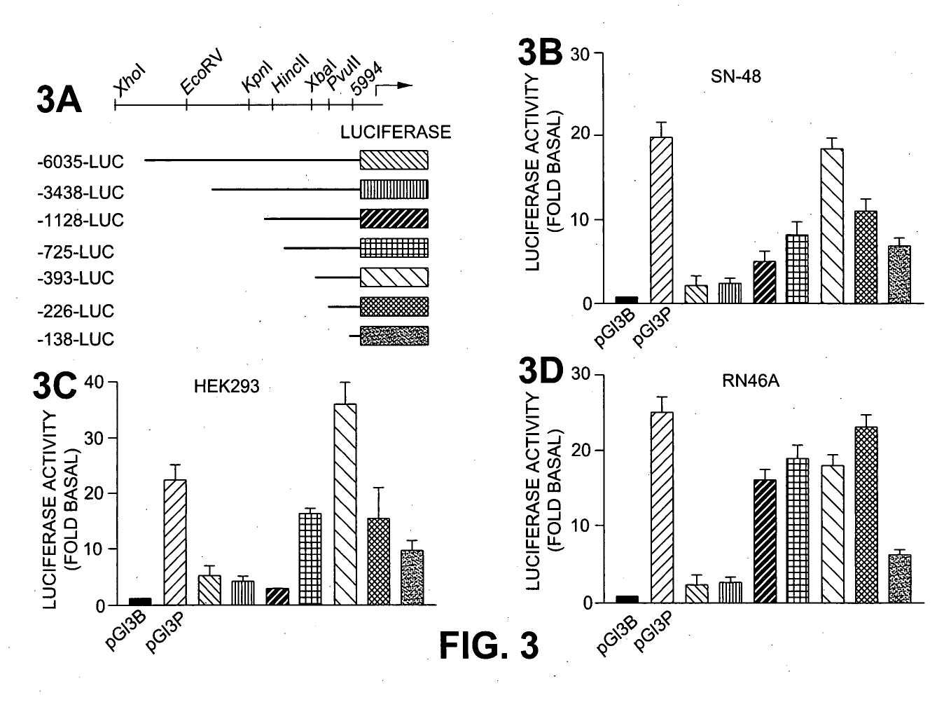

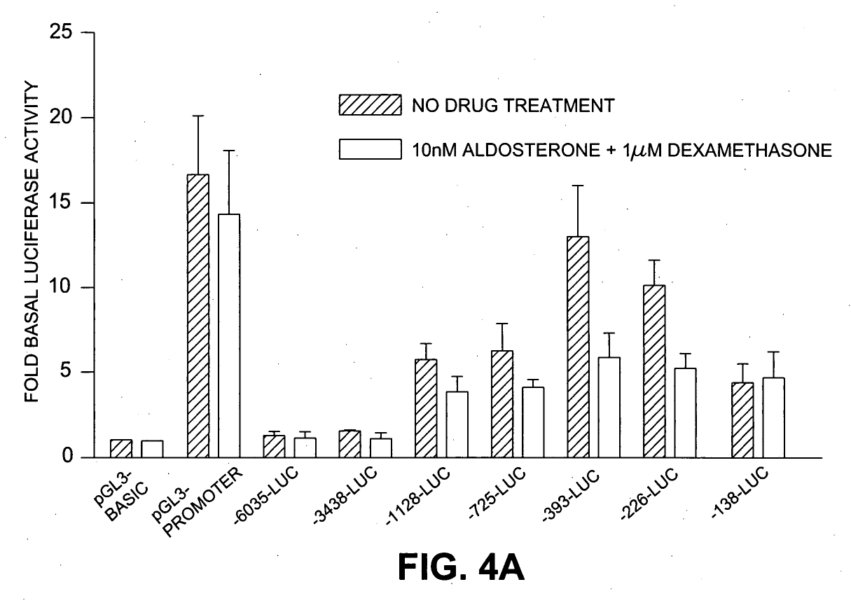

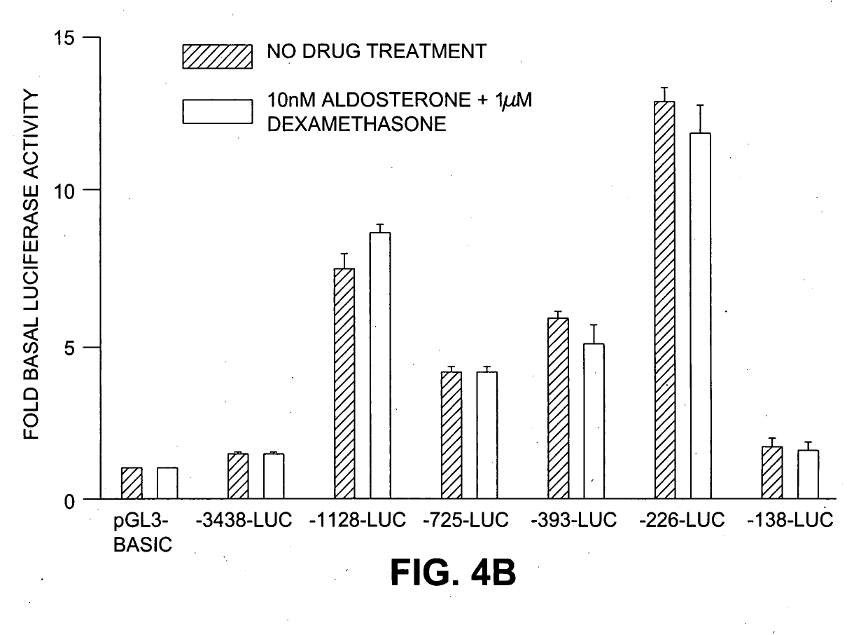

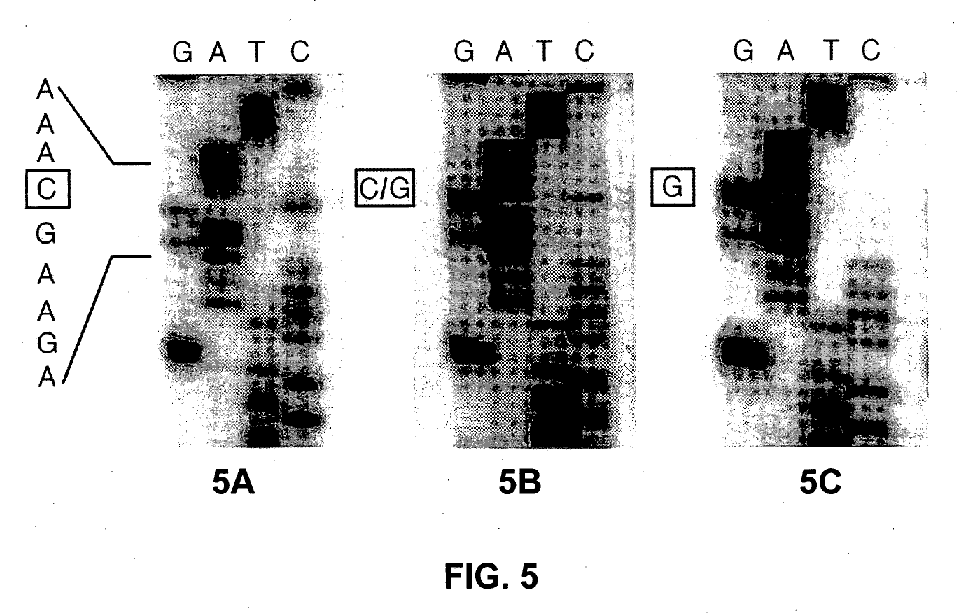

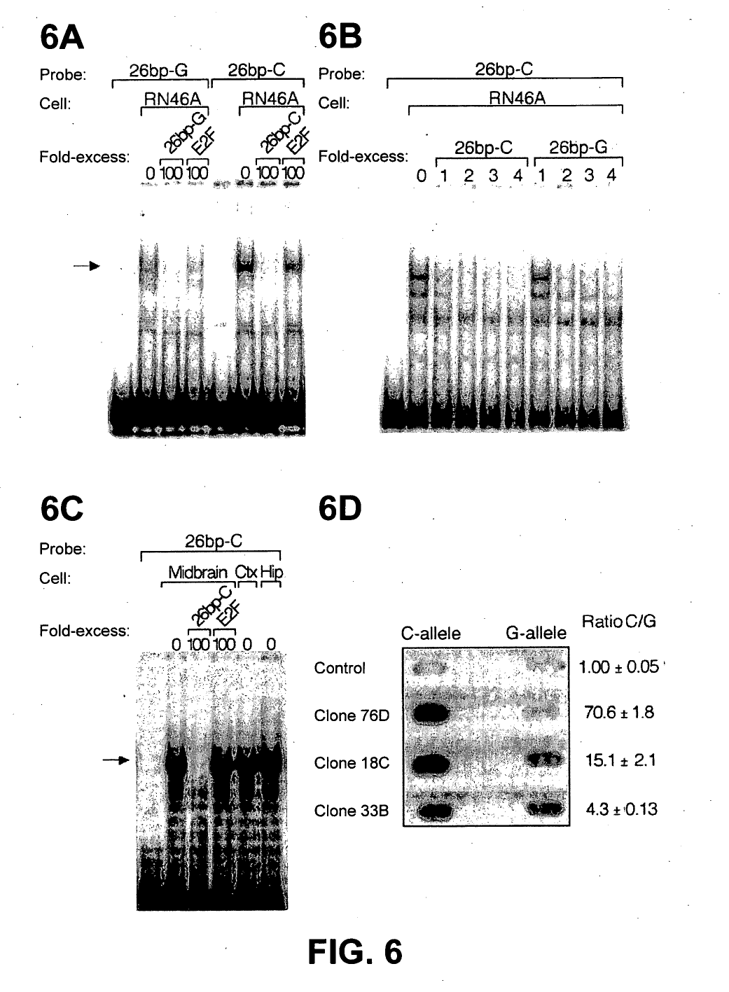

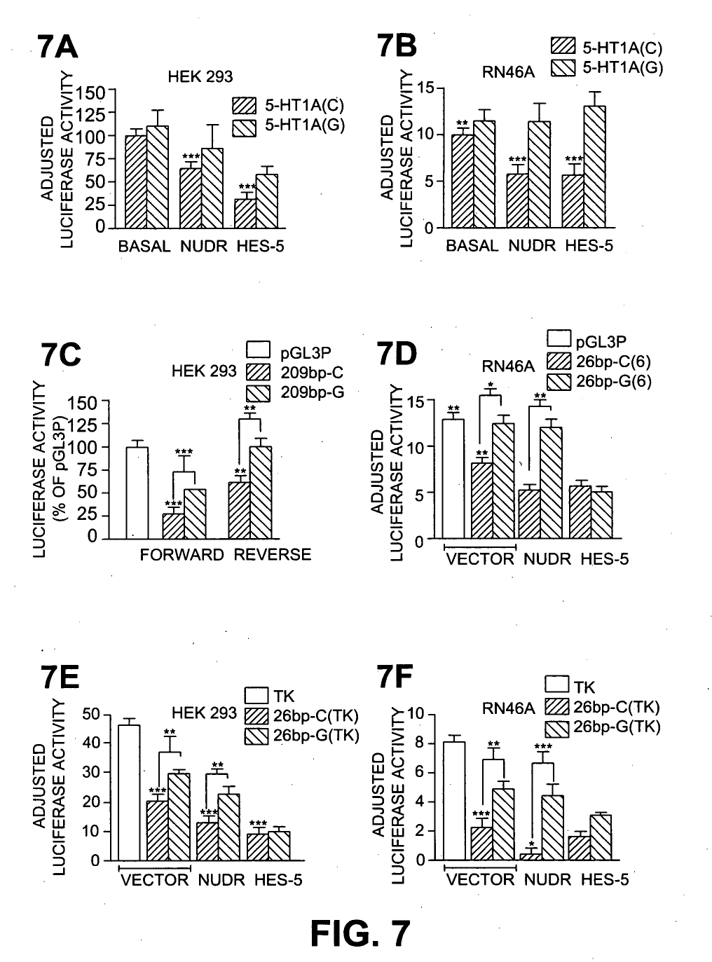

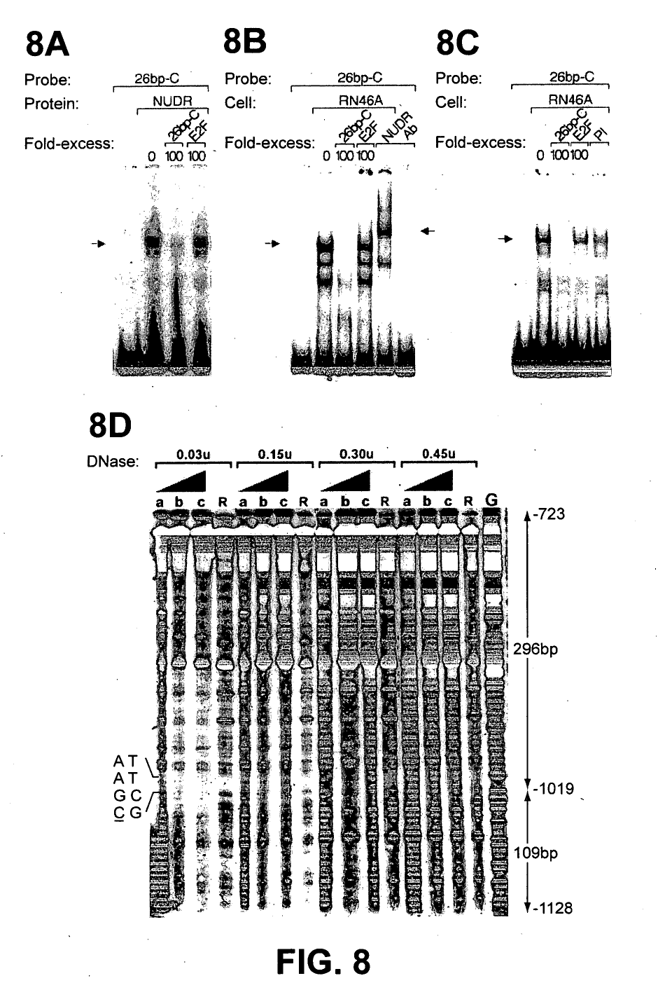

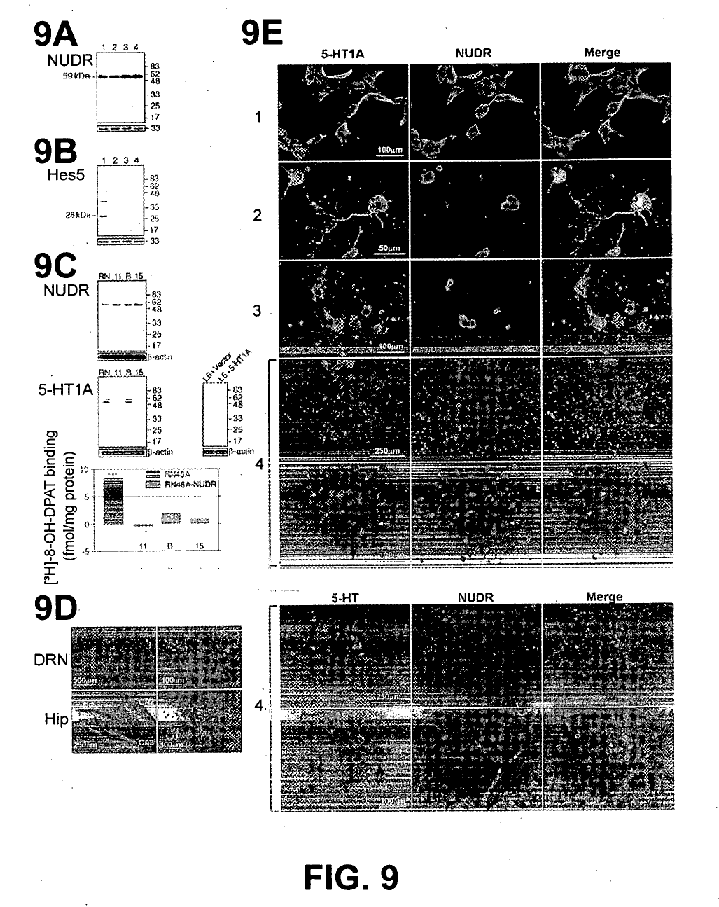

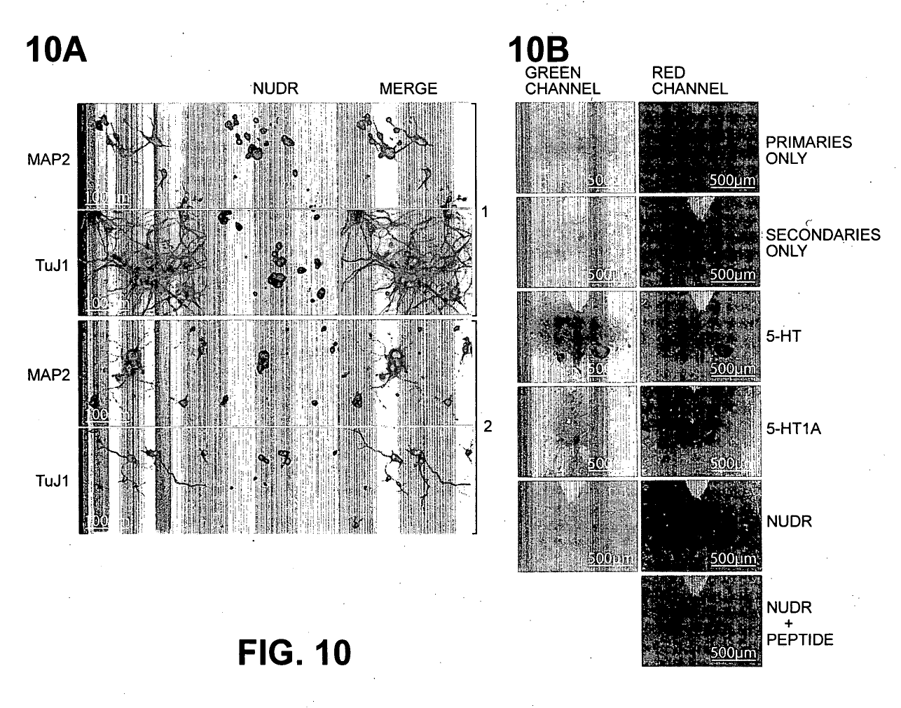

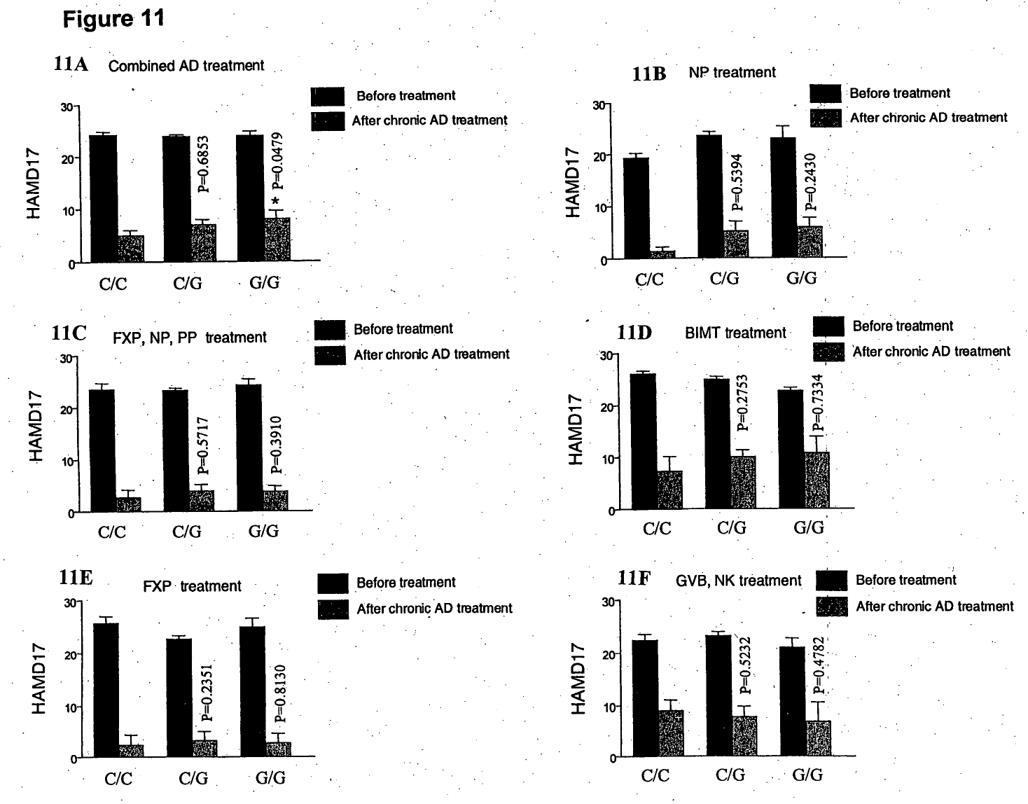

According to the present invention there has been identified a proximal ubiquitous promoter region of the 5-HT1A receptor gene. The promoter region is flanked by a repressor located between −1128 and −393 bp of the ATG codon in SN-48 cells. In RN46A cells repressor activity was located between −3438 and −1128 bp upstream of the ATG codon, suggesting cell-type specific regulation of the 5-HT1A receptor gene. As described above, SSRIs used to treat major depression appear to exhibit clinical effects upon the desensitization of the 5-HT1A receptor, ie. turning off of the 5-HT1A gene. This suggests that one of the abnormalities that could result in a tendency towards depression would be an elevated basal expression of5-HT1A receptors. As shown in FIG. 2, an excess of 5-HT1A autoreceptors would depress the firing of the raphe nucleus, reducing the release of 5-HT.

Thus, mutations in the region involved in basal repression of the 5-HT1A receptor gene would reduce or disrupt the repressor function of associated regulatory proteins leading to enhanced 5-HT1A autoreceptor expression and decreased serotonergic neurotransmission.

In one embodiment of the present invention a C-G change at −1019 bp was identified. According to the present invention, the occurrence of G at −1019 bp was found to correlate with patients with mental illness. Depressed patients were about twice as likely as the controls to have the homozygous G(−1019) genotype, whereas suicide victims were four times as likely to carry the same genotype. The association of the G(−1019) allele with major depression and suicide suggests that impaired repression of the 5-HT1A receptor could contribute to a predisposition towards unipolar depression and its most severe outcome.

Prior to the present invention there was no evidence for a clear genetic association of the 5-HT1A gene allele with a particular mental illness. From the results of the present invention, the identification of a C(−1019)G polymorphism, which is strongly associated with major depression and suicide, provides evidence of the use of this polymorphism as a genetic marker for mental illness. Increasingly, PCR-based gene detection is being used in prognostic and diagnostic evaluation of patients, and in criminological identification and characterization. For example, genetic testing of children of affected adults may allow for counseling or early treatment prior to development of an episode of major depression. In addition, the polymorphism can also be used to correlate the genetic change with properties such as severity or drug treatment response. In this aspect of the present invention a patient's response to certain drug therapies can be correlated to the presence or absence of the polymorphism. It therefore would be possible to check the patient for the polymorphism and based on this information determine what drug therapy would be best suited for that individual.

Thus, according to one aspect of this invention there is provided a DNA sequence containing a polymorphism in the repressor region of the 5-HT1A receptor gene characterized in that it reduces or disrupts the repressor function of a 5-HT1A regulatory region leading to enhanced 5-HT1A autoreceptor expression. In one embodiment there is provided a DNA sequence which contains a C-G change at −1019 bp in the repressor region of the 5-HT1A receptor gene. In a further embodiment of the present invention there is provided an imperfect palindromic sequence that flanks the C-G site. The palindrome has the sequence 5′-AACGAAGACNNNNNNGTCTTCTT-3′ (SEQ ID NO:2) (polymorphic site shown in double underline). In a further embodiment of the present invention the palindrome has the sequence 5′-AACGAAGACACACTCGGTCTTCTT-3′ (SEQ ID NO:3) (polymorphic site shown in double underline). The palindrome forms a structure that is recognized by DNA binding proteins.

The DNA sequence described above can be used, according to a further aspect of the present invention, as a diagnostic or prognostic marker for mental illness and behavioral disorders, as well as a predictive marker of behavioral traits. In this aspect of the invention the DNA sequence can be used as a probe in a diagnostic or prognostic test. The probe can be of any suitable length, as is well known in the art. A DNA probe ranging in length from about 10 to about 50 nucleotides would be suitable. The diagnostic or prognostic test could also include PCR amplification of the target sequence in a test sample, which is well know in the art. The primers used in the tests will of course flank the target sequence, one of such primers being for the sense strand and one other of such primers being for the anti-sense strand. An appropriately labeled DNA probe, as describe above could then be used to identify the target sequence in the test sample. The diagnostic or prognostic test could also include the sequencing of the target sequence in the test sample to identify the nucleotide sequence of the target sequence in the test sample.

In one example of this embodiment, the PCR primers were designed to amplify a 716 bp fragment of the human 5-HT1A 5′-flanking region from −1595 to −879 bp of the initial ATG codon. The sense primer had the following sequence: 5′-GTGGCGACATAAAACCTCA-3′ (SEQ ID NO: 4), and the antisense primer had the following sequence: 5′-TTCTTAAATCGTGTCAGCATC-3′ (SEQ ID NO: 5).

For a diagnostic kit, primers to amplify a smaller segment (e.g., 100-200 bp) surrounding the polymorphism would be designed and used in PCR of blood samples. The PCR products would then be analyzed by DNA sequence analysis, or by SSCP (Single Stranded Conformational Polymorphism) or PCR-based to probe for the polymorphism. Alternately, if the appropriate restriction enzyme becomes available, digestion with a restriction enzyme that differentiates between normal and polymorphic sequences could be used for the analysis of the PCR product. These assays would identify normal, heterozygous and homozygous alleles. Alternately, the repressor protein or specific antibodies that bind to the polymorphic site could be developed for use as an ELISA or radio-receptor competition assay for the presence of the polymorphism. Although rapid and efficient the competition binding assays may not be sensitive enough to discriminate between heterozygous and homozygous polymorphisms. Instead, it is proposed to use real time PCR and fluorescence detection of this purpose or other PCR-based methods.

In the context of the present invention the target sequence in the test sample will include the mutation in the repressor region of the 5-HT1A receptor gene. In one aspect of the invention the target sequence will include the −1019 bp locus.

The present invention further includes within its scope kits for the identification of the mutation, deletion or insertion in the repressor region of the 5-HT1A receptor gene. The kits will include a DNA sequence, as described above, to be used as a probe, together with other reagents required to complete the diagnostic or prognostic test. These reagents include but are not limited to DNA primers for PCR amplification of the target sequence together with reagents and enzymes required for PCR.

SSRIs are effective not only in treatment of major depression, but also of related mental illnesses that involve the serotonin system, such as bipolar depression, generalized anxiety disorder, obsessive-compulsive disorder, and panic disorder. Agents that directly modify the 5-HT1A receptor, such as agonists like buspirone, are effective in the treatment of generalized anxiety, and are beginning to be used for treatment of the negative symptoms of schizophrenia. Finally, agents that release serotonin (d-fenfluramine) are effective in eating disorders. One component of these illnesses could be abnormal regulation of the 5-HT1A receptor due to the polymorphic change at −1019 bp. Thus the identification of this polymorphism can provide a marker for sub-dividing the severity, phenotype, or treatment responsiveness of patients with these diseases.

Consistent with a functional role for the −1019 bp polymorphic region in regulation of the 5-HT1A gene, the present invention further comprises a protein or a protein complex from raphe nuclei that interacts with a fragment including the −1019 locus. In one embodiment of the present invention the fragment is a 26 bp segment that contains both the polymorphic site and a palindromic DNA sequence; however further fragments are included within the scope of this invention. In one embodiment of the present invention the proteins are transcription factors. In this embodiment of the present invention the transcription factors are selected from the group consisting of: NUDR/DEAF-1/suppressin and HES-5. This invention is the first reported evidence of the association of transcription factors with major depression and completed suicide.

According to the present invention there is provided a molecular mechanism by which the single nucleotide C(−1019)G polymorphism may regulate 5-HT1A gene expression in vivo by de-repression of the 5-HT1A promoter in pre-synaptic raphe neurons leading to reduced serotonergic neurotransmission. The C(−1019)G change dramatically impaired transcriptional repression of the 5-HT1A receptor gene by the identified trans-acting proteins NUDR and HES-5. Although both NUDR and HES-5 repressed the 5-HT1A receptor gene, the repressor activity of NUDR was the most sensitive to the C(−1019)G polymorphism associated with major depression and NUDR was the only factor capable of binding to the C(−1019) site in serotonergic raphe neurons. According to the present invention NUDR negatively regulates both 5-HT1A gene transcription and receptor expression. Importantly in the dorsal raphe nucleus and RN46A cells, NUDR protein is co-expressed with the 5-HT1A receptor and binds to the 5-HT1A promoter at the polymorphic TTCG site within the imperfect palindrome sequence, implicating NUDR in 5-HT1A regulation in vivo as well as in RN46A cells. Since RN46A cells were originally derived from E13 raphe neurons (White et al., 1994) and NUDR is also expressed in adult brain (LeBoeuf et al., 1998), NUDR may repress the 5-HT1A receptor gene throughout development and into adulthood. Because NUDR immunoreactivity is also present post-synaptically in 5-HT1A-expressing hippocampal and cortical neurons, de-repression should be induced by the G(−1019) allele. However NUDR can act as a repressor or an enhancer, depending on cell type and promoter sequence (Bottomley et al., 2001; Huggenvik et al., 1998). Interestingly, according to the present invention NUDR enhances, rather than represses 5-HT1A transcriptional activity in various hippocampal and septal cells (data not shown). Thus, although not wanting to be bound by any particular theory, it appears that the G(−1019) allele de-represses 5-HT1A transcription presynaptically, but may have the opposite effect to reduce NUDR-enhanced 5-HT1A transcription in post-synaptic cells. The net effect of these changes would be a reduction in serotonergic neurotransmission.

According to the present invention, the polymorphic site is in a region that has repressor activity in raphe cells, and the C-G change reduces this activity by inhibiting protein-DNA interactions. This leads to an enhanced expression of the 5-HT1A autoreceptor, which contributes to a greater predisposition towards major depression. Thus the protein or proteins that bind to the polymorphic region function as repressors of the 5-HT1A receptor, and constitute important drug targets for the development of novel therapeutic compounds to treat depression and related illnesses.

Thus according to this aspect of the present invention the “wild type” 5-HT1A sequence is used to identify proteins that bind to the repressor region. These naturally occurring proteins could then be modified so as to improve their protein-DNA interactions in the mutated repressor region, to thus mimic the normal binding.

In addition, novel therapeutics, based on improving the protein-DNA binding could be identified using the mutated repressor region of the present invention. Thus this invention is also directed to a method of identifying novel therapeutics using a DNA sequence containing a mutation in the repressor region of the 5-HT1A receptor gene, to identify therapeutics that have an improved protein-DNA binding capability with the mutated repressor. Novel therapeutics that act to enhance the expression or activity of the repressor protein would also be covered by the present invention.

Novel therapeutics could be identified using a number of known techniques. For example an oligonucleotide incorporating the repressor DNA element could be used to screen a cDNA expression library and clone cDNA's of proteins that bind to the oligonucleotide in a specific manner. Also an oligonucleotide incorporating the repressor DNA element cloned upstream of a reporter gene could be used to screen a cDNA library fused to the appropriate activation domain for the reporter gene in yeast or mammalian one-hybrid approach. Alternatively the repressor element could be used to generate reagents for the purification of the binding proteins that interact with that element. The repressor element could also be used as a probe to follow the purification of proteins that interact with the element.

The DNA sequences of the present invention can also be used to develop mimetics of the DNA binding domain of the repressors that can inhibit competitively the activity of proteins that bind to the repressor region in cases where it is important to reduce the DNA-protein interaction (eg., in hyper-aggressive patients or patients with related mood disorders). Alternately, the DNA sequence could be used to develop oligonucleotide analogous to the binding site to squelch the activity of proteins that bind to the repressor region. In addition, novel therapeutics that reduce the expression or activity of the repressor proteins would also be covered by the invention.

As previously discussed, within the proximal 5′-flanking region of the human 5-HT1A receptor a novel glucocorticoid responsive region that suppresses reporter gene expression has been identified. In SN-48 cells, a model of post-synaptic 5-HT1A expressing neuron, dexamethasone pretreatment suppresses the expression of the 5-HT1A receptor gene by acting at a glucocorticoid-responsive element located from between −393 bp and the ATG initiation codon, and more specifically from about −226 bp to about −138 bp from the initial ATG codon. This element differs from previously-described GRE sequences and thus represents a novel glucocorticoid element.

The glucocorticoid-mediated repression of the 5-HT1A receptor is another mechanism by which the expression of the receptor may be regulated. For example, a large proportion of depressed patients have attenuated response to dexamethasone suppression, symptomatic of reduced glucocorticoid responsiveness and leading to elevated levels of glucocorticoids. Alteration in the DNA sequence that mediates glucocorticoid regulation could lead to abnormal over-expression of the 5-HT1A receptor. Such alteration in the glucocorticoid response region of the 5-HT1A gene may be prognostic of patients that respond to glucocorticoid therapy in combination with anti-depressant compounds.

The present invention is illustrated in the following examples, which are not to be construed as limiting.

### EXAMPLES

### Methods

Construction of Luciferase Reporters

The luciferase plasmid −6035-luc was obtained by subcloning the 5′-flanking SalI/BssHII 6-Kb fragment of the human 5-HT1A receptor gene into the XhoI/MluI site of a modified pGL3-Basic vector (Promega) containing a repeated KpnI/SmaI cassette in the reverse orientation. From −6035-luc, all subsequent constructs were generated. The −3438-luc and −226-luc were constructed by digestion with EcoRV and PvuII respectively, followed by internal ligation. The −1128-luc was obtained by insertion of a KpnI/BssHII fragment into pGL3 -Basic vector (Promega) digested with KpnI and MluI. Digestion with HincII and SmaI generated a fragment that was inserted into the SmaI site of pGL3-Basic to produce −725-luc construct. Similarly, the DNA segment obtained by digestion with XbaI and NheI was inserted into the NheI site of pGL3-Basic and was called −393-luc. Finally, PCR amplification of a proximal 164-bp fragment gave a product that was then digested with HindIII and subcloned into a SmaI and HindIII digested pGL3-Basic vector to generate −138-luc. All plasmids were purified by CsC1 equilibrium gradient centrifugation and quantified spectrophotometrically (Ausubel et al., 1989).

Cell Lines and Transient Transfections

Mouse septal-neuroblastoma SN-48 cells and human embryonic kidney cells HEK293 were grown in Dulbecco's modified Eagle medium (DMEM) (Gibco BRL) supplemented with 10% v/v heat-inactivated fetal bovine serum (FBS) at 37° C. in 5% CO₂. SN-48 cells were differentiated by reduction of FBS to 1% v/v and treatment with 10 μM retinoic acid. The rat raphe RN46A cells were cultured in Neurobasal medium (Gibco BRL) supplemented with 10% v/v heat-inactivated FBS and 0.5 mM 1-glutamine at 33° C. in 5% CO₂.

SN-48 and HEK293 cells were transfected in 10 cm dishes using the calcium phosphate co-precipitation method (Ausubel et al., 1989). To correct for differences in transfections efficiencies between dishes, 2 μg of pCMV-βgal plasmid were co-transfected with 20 μg of luciferase reporter constructs. After 14-16 hours incubation with CaHPO₄, cells were passage into three 3.5 cm dishes, and incubated for 36 hours with fresh medium containing penicillin (50 U/ml) and streptomycin (50 μg/ml) before assaying for luciferase activity. SN-48 cells were differentiated during this period of time and if applicable, treated 12 hours prior to harvest with 10 nM aldosterone and 1 μM dexamethasone in DMEM supplemented with 1% heat-inactivated charcoal-treated serum.

The embodiments of the invention in which an exclusive property of privilege is claimed are defined as follows:

1. A DNA sequence containing a mutation in the repressor region of the 5-HT1A receptor gene wherein said mutation results in a reduction in repressor function leading to enhanced 5-HT1A receptor expression.

2. The DNA sequence of claim 1 wherein the mutation is located in the region from about −3438 to about −393 from the ATG codon of the 5-HT1A receptor gene.

3. The DNA sequence of claim 2 wherein the mutation is selected from the group consisting of: a single or multiple base pair change, an inversion, a deletion and an insertion.

4. The DNA sequence of claim 3 wherein the mutation is a G-C change at −1019 bp from the ATG codon of the 5-HT1A receptor gene.

5. The DNA sequence of claim 4 wherein the sequence is AACGAAGACNNNNNNNGTCTTCTT.

6. The DNA sequence of claim 5 wherein the sequence is AACGAAGACACACTCGGTCTTCTT.

7. A glucocorticoid-responsive element located from between −393 bp and the ATG initiation codon of the 5-HT1A receptor gene.

8. The glucocorticoid-responsive element of claim 7, wherein said element is located from about −226 bp to about −138 bp from the initial ATG codon.

9. A method for detecting depression and related mental illnesses comprising the steps of:
selecting primers to amplify a DNA region from the repressor of the 5-HT1A receptor gene;
amplifying the DNA region from the repressor of the 5-HT1A receptor gene; and determining the sequence of the DNA region from the repressor of the 5-HT1A receptor gene, whereby identifying a mutation in said DNA region, wherein said mutation results in a reduction in repressor function leading to enhanced 5-HT1A receptor expression, which is correlated to depression and related mental illnesses.

10. The method of claim 9 wherein the mutation is located in the region from about −3438 to about −393 from the ATG codon of the 5-HT1A receptor gene.

11. The method of claim 10 wherein the mutation is selected from the group consisting of: a single or multiple base pair change, an inversion, a deletion and an insertion.

12. The method of claim 11 wherein the mutation is a G-C change at −1019 bp from the ATG codon of the 5-HT1A receptor gene.

13. A method of identifying a protein which binds to a DNA region from the repressor of the 5-HT1A receptor gene comprising the steps of:
using an oligonucleotide from the repressor region of the 5-HT1A receptor gene to screen a cDNA expression library;
identifying a protein that binds to the oligonucloetide; and
cloning cDNA's of the proteins that bind the oligonucleotide.

14. The method of claim 13 wherein the repressor region is from about −3438 to about −393 from the ATG codon of the 5-HT1A receptor gene.

15. The method of claim 14 wherein the oligonucleotide from the repressor region includes the −1019 bp position from the ATG codon of the 5-HT1A receptor gene.

16. The method of claim 15 wherein the oligonucleotide includes the sequence TTCG.

17. A method for identifying a therapeutic effective agent to treat depression and related mental illnesses, wherein said agent binds to a DNA sequence containing a mutation in the repressor region of the 5-HT1A receptor gene and said mutation results in a reduction in repressor function leading to enhanced 5-HT1A receptor expression, comprising the steps of:
screening a library of test agents;
identifying an agent that binds to the DNA sequence.

18. The method of claim 17 wherein the mutation is located in the region from about −3438 to about −393 from the ATG codon of the 5-HT1A receptor gene.

19. The DNA sequence of claim 18 wherein the mutation is selected from the group consisting of: a single or multiple base pair change, an inversion, a deletion and an insertion.

20. The DNA sequence of claim 19 wherein the mutation is a G-C change at −1019 bp from the ATG codon of the 5-HT1A receptor gene.

21. An antibody to a protein, or epitope thereof, wherein said protein binds to the DNA sequence as defined in claim 1.

22. The antibody of claim 21, wherein the protein is a transcription factor.

23. The antibody of claim 22, wherein the transcription factor is selected from the group consisting of NUDR/DEAF-1/suppression and HES-5.


SEQUENCE LISTING (8 sequences)

SEQ ID NO: 1 - DNA, 3045 bp, human
atcatcaataatatccgttataaagcttgcttttctttaggttaactttagaggccttgaagaataagagctcatctctttacaggagctttggtttgcagcatttacttaagaaatatttggtattctgtatctttaagagttaaacatagaagaattggctaagtgaaaatgaatgaaacgcaatatcattctgcatatatcatttattatatatcacagtattattagttttaaaagttaaacataaatatctattatgycattgsacgaytaggysaacctartcrgtgctgcgaatactttcgatacttctgtttccctcctagtattcataagtgtgcctttgaaaacgttttaaattgtaagaaataaaatgtttgatatattatgtatattattactaagaaaaaacttgaattactttggattttgaaaaactttgataaattctacatcatagcatattgaagcaagaataacaaatgctatacctcaggaatattaattccagattttacagcattttaactttcttgatgagaaaaaataaatttgtcagttattaaactatttggatccaacagatgaaagcagaattctaactaacatatttattgatttatttgtgatttacatatttacatgtgttgtttgacacaattcttaattatgttcttgatatgcatatatttgcttcttaaattttaagtttcctttattttactttgtttatagtctcaactataatttcaaagtttaattttagataattcagccttttaaatattttcccattataatttttgtgacctctaactctattttaactgtaaatatagttctgtatttgtgaagagactttagaagtggaaatagataccttcacaaatcttaaaagacttcttcagagtctgtaaacagcattaccatgtatacttatctctttctttgcatgccatgatcatcacaatgcatggctcatgtggtggcatgctgaatgattgagtgggactgtgccagctgaactataaaaaaaaaaaacaaacaaaaccttatccaaacacactgtcctgtattgtaatgcattggcccaactggattctttttgatgctttggtgattgctcttttgtttgggcttggagaattcagagctatgaaattcagagctcagatttgaacacaatattaagattattgcaatctgtagtgaatctgttcatgttatccagtgtcaactgcttttgagattgcattcctttcacctcaggcatgcaatcaggatgtataagtgaaatgttgtgtggtatgtttactgtagttgcttagaagtccattctttaccaatgctcaaatgtgattaaatttgttttcttgttaaaggaaacagcttagaacaaacccttgtaagtatctttatttcagtgatttaacatttccaaatgttaaatcatttggaaaatgcaatactattcgtttctccaacaaaaggtaaatttatgtcagttccaaagttcaggttatgacagcacaaaaccaacacaggtgaaagtgttagcctagctttattaaaatggcattcccagttagaacttgtgaatgacagatacttcaggctttcgaaggaagctaaaacatataataggcctgatatataaggttcagagcaaaagagggcactaaaataaatttttaaagaaaataggaaggagacaaaactcaatactaccttgtcttttaataactgtcttcctctttctaaaagttgttgtatttcctcaatacttgcttcatttctggcataagggtttccagatggcactctaaaacatttgccagaaggtggcgaacataaaacctcattgcttagaactgtcccaggtgctgaacccagtttctgagattaagagaggctagccggctagcgaaccgggattccaccaagtttcccccagaggtttgcaggctctggtaagaagtgcaaaaggccatgtgaaatgccaggcttcacttagaacacatatgcaaaatatttccatccctgaatttactagccacaaagctatgggaagtggcagtgtcactgaaattacaagtgtagtagtgatggaaaagtgtgtgtgtgtttagaatatatatcacactgagttttgttcttcatttcgagatgcagttgtttacctctccttgtcctttgacacgtcctttataatttcgttctctcccggttccccaacgttaaaaaaaaagtcacaggcaatattctccctgagggagtaaggctggactgttagatgataacggaggtaccgttttgttgttgttgtcgtcgttgttcgtttgtttttggagacggagtctcgctctgtcgcccaggctggagtgcaatggcgcgagaacggaggtagctttttaaaaacgaagacacactcggtcttcttccatcaattagcaataattgggagactgacccaggactgttcaccttcccattcaggctccctatgcttccttttctcatctcctattgccactctgggatgctgacacgatttaagaatttggcagataatatgaggcaaggagtagttggaattccctcccccaagtttttccaaccccagttttgctgggttggaggcggagtttatttgttacaaccttggtctgaccggcaggatctggtgtgtgtaagtgagttctgagtctctgttgacaaaaagagactcgaatgcaaagacgctgagctagagggagaggagggcggggacccagaggaaagaggcactcctcggggttggggaagtattaggaggggagggttagagtgggagggaaggagcctggctttcgaagcgactcacagagggataaataaagggaagtgaggaggaagagggagactgaaagggaaggcaggtggggagaagggggacgaaagaggcagaagagagagaagagaggaggagagagggggagagagggaaggaaggaaatagggagaggagggtcacagagtgaccgtggaggatggggcttctcg

SEQ ID NO: 2 - DNA, 24 bp, human
  n=a, c, g, or t
aacgaagacnnnnnnngtcttctt

SEQ ID NO: 3 - DNA, 24 bp, Artificial Sequence
  Description of Artificial Sequence Specific Palindromic Sequence of SEQ ID NO2
aacgaagacacactcggtcttctt

SEQ ID NO: 4 - DNA, 19 bp, Artificial Sequence
  Sense Primer
gtggcgacataaaacctca

SEQ ID NO: 5 - DNA, 21 bp, Artificial Sequence
  Description of Artificial Sequence antisense primer
ttcttaaatcgtgtcagcatc

SEQ ID NO: 6 - DNA, 24 bp, human
  Sense Primer
aacgaagacacactcggtcttctt

SEQ ID NO: 7 - DNA, 26 bp, human
  Antisense Primer
ggaagaagaccgagtgtgtcttcgtt

SEQ ID NO: 8 - DNA, 21 bp, rat
  unrelated rat E2F sequence
atttaagtttcgcgccttttc